(19) United States
(12) Patent Application Publication
(10) Pub. No.: US 2019/0388024 A1
(43) Pub. Date: Dec. 26, 2019

(54) DEVICE TO DETECT; ASSESS AND TREAT SNORING, SLEEP APNEAS AND HYPOPNEAS

(71) Applicant: WHISPERSOM CORPORATION, Annapolis, MD (US)

(72) Inventors: David Brian Goldstein, Groton, MA (US); Michael G. Nathans, Annapolis, MD (US)

(73) Assignee: WHISPERSOM CORPORATION, ANNAPOLIS, MD (US)

(21) Appl. No.: 16/532,213

(22) Filed: Aug. 5, 2019

Related U.S. Application Data
(63) Continuation of application No. 14/535,561, filed on Nov. 7, 2014, now abandoned.
(60) Provisional application No. 61/901,668, filed on Nov. 8, 2013.

(51) Int. Cl. A61B 5/00; A61B 5/11; A61B 5/0205

(52) U.S. Cl. CPC A61B 5/4818; A61B 5/4836; A61B 5/6898; A61B 5/7405; A61B 5/1495; A61B 5/002; A61B 5/11; A61B 5/0205; A61B 5/7455

(57) ABSTRACT

A Device to detect, assess and treat Snoring, Sleep Apneas and Hypopneas. The present invention (the “Invention”) relates to an apparatus to detect, assess and end occurrences of snoring, sleep apnea events and hypopnea episodes, in a manner that will decrease or eliminate hypoxia, hypercapnia and the disturbance of cardiac and pulmonary hemodynamics, and give users of the apparatus a report of their critical sleep data each morning.

### CROSS-REFERENCE TO RELATED APPLICATIONS

This application is a Continuation of U.S. patent application Ser. No. 14/535,561 filed Nov. 7, 2014, which claims the benefit of 61/901,668 filed Nov. 8, 2013. All of the foregoing are incorporated by reference in their entireties.

### DESCRIPTION

A Device to detect; assess and treat Snoring, Sleep Apneas and Hypopneas.

### TECHNICAL FIELD

The present Invention (the “Invention”) relates to an apparatus to detect, assess and end occurrences of snoring, sleep apnea events and hypopnea episodes, in a manner that will decrease or eliminate hypoxia, hypercapnia and the disturbance of cardiac and pulmonary hemodynamics, and give users of the apparatus a report of their critical sleep data each morning

### BACKGROUND ART

Sleep apnea and hypopnea are breathing disorders that occur during periods of sleep. It is an intermittent cessation or reduction of ventilation during sleep that results in a decrease in blood oxygen levels (hypoxia), increase in CO2 (hypercapnia), and vasoconstriction. The long term effects of these physiological changes are associated with the development of cardiac arrhythmias, congestive heart failure, cardiac ischemia, hypertension, heart disease, brain and liver damage, diabetes and cognitive impairment. An apnea is defined as a =>90% reduction in airflow for longer than 10 seconds. Hypopnea is overly shallow breathing (=>30% reduction in airflow lasting 10 seconds or longer and an associated 4% (or greater) reduction in the patient's blood oxygen levels, and it results in arousal—waking the patient up.

The causes of the various forms of Sleep apnea and hypopnea are not fully understood.

There are three general types of sleep apnea. Obstructive, Central and Mixed.

Obstructive Sleep Apnea (OSA) is the most common type and is a blockage or occlusion of the oropharyngeal (upper) airway due to a loss of patency of its muscles. With OSA, respiratory functions continue as paradoxical movement of the thorax or abdomen. The cause or causes of OSA is still a matter of much debate and research. The average apnea event lasts 20 seconds however events of 2 to 3 minutes are also known to occur. During the event, a number of physiological events also occur. These include a vagal bradycardia, an increase in blood pressure, an increase in norepinephrine, and paradoxical respiratory efforts with increased respirator efforts. As an apnea event progresses, there is an increasing effort to breathe, increasing carbon dioxide (hypercapnia), decreasing oxygen, and increasing level of proprioception. The longer the apnea event, the more extreme these changes are.

At the end of an apnea event, tone (patency) returns to the upper airway muscles so that the upper airway suddenly re-opens. This can be associated with a sudden gasp or choking as air rapidly enters the lungs, and surges in heart rate and blood pressure take place.

Following are excerpts parsed from various research papers describing apneas, hypopneas and. the accompanying physiological events that also occur.

It has been believed that an arousal from a deeper stage of sleep to a lighter stage of sleep was required to terminate an apnea episode. However, studies have cast doubt on that assertion: summary, in the vast majority of patients, if not in all patients, arousal is required neither to initiate UA (Upper Airway) opening nor to obtain adequate flow. UA opening would occur at approximately the same time regardless of when or whether arousal occurs and the flow response in most patients would still be timely and adequate. Arousals are incidental events that occur when the thresholds for arousal and arousal-independent opening are close to each other, as they appear to be in patients with OSA. By promoting an unnecessarily high flow response at UA opening, arousals help perpetuate cycling and likely exacerbate OSA.” (Role of Arousals in the Pathogenesis of Obstructive Sleep Apnea. Younes, Magdy. American Journal of Respiratory and Critical Care Medicine: Mar. 1, 2004. Which is hereby incorporated by reference.)

Although cortical activation is the gold standard for definition of arousal, several studies show there are different levels of central nervous system activation. At the lower range of arousal responses are those inducing reflex motor responses, autonomic activation, and appearance of slow wave EEG activity, i.e., delta bursts (D-bursts) and K-complex bursts (K-bursts), all defined as “subcortical arousals.” At the upper range are arousal responses implying a cortical activation represented by MA6 and phases of transitory activation (PAT).

These findings might corroborate the hypothesis of the existence of two separate neural systems integrated in the arousal network and undergoing different modulatory influences. Further studies indicate that overall, increasing ventilatory effort may be the most important stimulus to arousal from sleep, and the stimulus to arousal from hypoxia and hypercapnia may be mediated principally through stimulating an increase in ventilatory efforts.

These considerations raise the question of possible manipulation of the arousal response to maximize the beneficial effects related to facilitating resumption of airflow, but minimize the adverse consequences related to sleep fragmentation and post-apnea hyper ventilation. These latter effects appear to relate more to cortical than brainstem arousal.

Furthermore other studies concluded that: “The current findings suggest that strategies of induced arousal, at an intensity level stimulating respiration while avoiding recruitment of the ascending arousal system and its potential effects of sleep disruption, could have potential application as a therapeutic modality.” “Apnea was detected by tracheal breath sounds which were picked up by microphone . . . stimulation decreased the frequency of apnea episodes and the longest apnea duration. This resulted in . . . an increase in arterial oxygen saturation. Moreover . . . stimulation decreased sleep stages I and II, and increased stages III and IV, These findings suggest that . . . stimulation using the apnea demand-type stimulator may be an effective treatment for OSA.”

(A New Treatment for Obstructive Apnea Syndrome by Electrical Stimulation of Submental Region

Hiroshi Miki, Wataru Hida, Hiroshi Inoue And TarnotsutAkishimma

The First Department of Internal Medicine, Tohoku

University School of Medicine, Sendai 980

Tohoku J. exp. Med., 1988, 154, 91-92

Which is hereby incorporated by reference.)

Other research has determined that: “ . . . the Psa (Blood Pressure) and HR (Heart Rate) increased more and the SV (Stroke Volume) decreased more in the apnea that was terminated by an EEG (cortical) arousal compared with the apnea without an EEG (subcortical) arousal.”

(Arterial blood pressure responses to graded transient arousal from sleep in normal humans

R. J. O. Davies, P. J. Belt, S. J. Roberts, N. J. Ali, and J. R. Stradling

Osler Chest Unit Churchill Hospital, Headington, Oxford 0X3 7LJ, United Kingdom

J. Appl, Physiol. 74(3): 1123-1130, 1993.

Which is hereby incorporated by reference.)

Furthermore externally applied stimulus is reported to cause a “ . . . trend among our subjects to shortening of the apnea immediately after the stimulated apnea; that is, the effect of the tone appeared to extend to the next apnea. We would hypothesize that the acoustic stimuli did alter sleep state and thus arousal threshold such that the immediately succeeding apnea might have been more susceptible to concurrent respiratory afferent stimuli”.

(Effect of induced transient arousal on obstructive apnea duration R. C. Basner, E. Onal, D. W. Carley, E. J. Stepanski and M. Lopata.

Department of Medicine, Section of Respiratory and Critical Care Medicine, University of Illinois at Chicago College of Medicine, and the Department of Veterans Affairs West Side Medical Center and University of Illinois Hospital, Chicago, Ill. 60612 0161-7567/95 Copyright @ 1995 the American Physiological Society. Which is hereby incorporated by reference.)

This favorable response to audio acoustic stimulus took place in spite of the trend for Obstructive Sleep Apneas to increase in both frequency and duration during a nights sleep.

The kind of stimuli provokes different responses in human subjects: “Previous studies using single-modality paradigm have shown that sensory gating systems, which select relevant sensory information, remain functional during sleep. In humans, relevant stimuli (e.g. sound >65 dB, one's own name, experimental noxious stimulation) induce arousal response more frequently and results in more intense response compared with irrelevant stimuli . . . . Simultaneous multi-modality sensory inputs from body surface and from other organs (e.g. ear) not only increase the amount of sensory inputs but also can maximize the relevance of stimuli”. (Halasz et al., 2004; Kisley et al, 2001; Velluti, 1997. Which is hereby incorporated by reference.)

Central Sleep Apnea results from the brain failing to signal the muscles to breathe. The neural drive to the respiratory muscles discontinues for a brief period of time. These transients may continue throughout the night for periods from ten seconds to as long as 2 to 3 minutes. The physiological effects are similar to those of Obstructive Sleep Apnea.

Mixed Sleep Apnea is a combination of Obstructive Sleep Apnea and Central Sleep Apnea.

There are several known treatments for sleep apnea. They consist of physical, electrical, and. mechanical methods, surgery, and attempts at pharmacological treatment. The treatment regimen is tailored to the individual, and is based on the medical profile of the patient being treated.

The most common effective treatment for patients with sleep apnea is nasal continuous positive airway pressure (CPAP). in this form of treatment; the patient wears a mask over the nose while sleeping. The mask is connected to a compressor that creates a positive pressure in the nasal passages. The continuous positive airway pressure system prevents the airway from closing or becoming obstructed during sleep. The air pressure from the continuous positive airway system is constant, and can be adjusted to best suit the individual's apnea condition. The air pressure in the continuous positive airway pressure system must be adjusted so that it maintains an open airway in the patient during all periods of sleep, but does not provide excessive pressure such that the device is bothersome to the patient. U.S. Pat. No. 4,655,213 discloses sleep apnea treatments based on the principles of continuous positive airway pressure. There have also been recent attempts at varying the applied pressure to increase the effectiveness of continuous positive airway pressure treatment. U.S. Pat. Nos. 4,773,411 and 6,539,940 disclose such techniques. The disclosures of these United States patents are incorporated herein by reference.

Another treatment for sleep apnea in certain patients involves the use of a dental appliance to reposition oral structures such as the tongue and the lower jaw. This form of treatment is typically performed by a dentist or dental specialist such as an orthodontist.

Surgery has also been performed to treat sleep apnea. In some surgical treatments, the size of the airway is increased and/or the width of the tongue is decreased, These surgical procedures contain elevated levels of risk in comparison to other treatment methods, and often times are not entirely effective or their effectiveness fades over time. The form of surgery to be undertaken is specific to the patient and the patient's medical profile. The removal of obstructive tissue in the airway such as adenoids, tonsils or nasal polyps is a common form of surgical treatment for sleep apnea. The surgical correction of structural deformities is also a common form of surgical treatment for sleep apnea.

Another form of surgical treatment for sleep apnea is uvalopalatopharyngoplasty. This procedure removes excess tissue from the back of the throat, such as tonsils, uvula, and part of the soft palate. Somnoplasty is also being investigated as a possible treatment for sleep apnea. Somnoplasty uses radio waves to reduce the size of some airway structures such as the uvula and the back of the tongue.

Other forms of surgical intervention for sleep apnea include maxillo-facial reconstruction. Another form of surgical treatment for patients with severe and life threatening sleep apnea is Tracheostomy. This procedure involves making a small hole in the windpipe that accommodates a tube. The tube is opened only during sleep, and allows a patient to take air directly into the lungs, effectively bypassing any upper airway obstructions. Tracheostomy is an extreme procedure that is very rarely used except for cases of imminent life threatening sleep apnea.

Attempts at pharmacological treatment for sleep apnea have included respiratory stimulants such as theophylline, acetazolamide and medroxyprogesterone, and adenosine. Drugs that stimulate brain or central nervous system activity, such as naloxone and doxapram, have also been used in an attempt to treat sleep apnea. Other drugs that act on the neurotransmitters involved with respiration have also been used in an attempt to treat sleep apnea. These drugs include serotonin, dopamine, tryptophan, fluoxetine, and others.

More recently, systems have been developed for the purpose of clearing upper airway passages during sleep using the electrical stimulation of nerves or muscles. In some cases, these systems require surgical implantation of sensors and associated electronics that detect when breathing has ceased and then stimulate the breathing process. Some hybrid systems have been developed that require surgical insertion of one or more sensors plus external equipment for monitoring the breathing process or removing the obstruction when breathing ceases.

Another treatment option is oral pressure therapy (OPT). The system consists of three main components—a vacuum console; a soft, flexible mouthpiece; and a tubing set. The mouthpiece is worn inside the mouth during sleep and is attached to the console with the tubing set. The user places the mouthpiece in their mouth, which is attached to the console via the tubing set. Once the mouthpiece is in place, the console will apply a light vacuum to gently draw the tongue and soft palate forward, which actively opens the airway.

And finally there is Nasal Expiratory Positive Airway Pressure (EPAP). These disposable devices attaches over the nostrils with a hypoallergenic adhesive. During inhalation, a valve opens, allowing the user to breathe in freely. When exhaling, the valve closes and air passing through the nose is directed through two small air channels. This increases the pressure in the airway, maintains pressure and helps to keep the airway open until the start of the next inhalation.

An apparatus has been patented a means for detecting the onset of a sleep related disorder using pulse rate and blood oxygen content information as measured by the device; U.S. Pat. No. 7,387,608 discloses sleep apnea treatments based on those principles. The disclosures of these United States patents are incorporated herein by reference.

An apparatus has patented a means for detecting the onset of a sleep related disorder using a multiplicity of microphones. Said apparatus has the microphones emplaced within a collar worn around the neck of the patient. The apparatus detects breathing sounds, and in an embodiment when it detects breathing that is “substantially different from said recorded at least one signal pattern that is associated with a normal breathing pattern of said person; and creating a stimulus to said person's neck muscles to cause said person to move said person's neck muscles to move said person's head backwards to restore normal breathing before cessation of breathing occurs”, as disclosed in U.S. Pat. No. 6,935,335. The disclosures of these United States patents are incorporated herein by reference.

Patent application 20100076251 discusses, among other things, an apparatus and method for detecting respiration information of a patient and for providing a stimulation to the patient. A sleep sensor transducer includes a pyro/piezoelectric film. A first and second electrode can attach to the film to transmit the detected respiration information to a closed loop neuromodulator and to receive stimulation energy from the closed loop neuromodulator to provide the stimulation. The disclosures of these United States patents are incorporated herein by reference.

Patent application 20100063350 discloses a system and method for anti-habituating sleep therapy using a closed loop neuromodulator. A first sleep disorder event can be detected using first activity information, and a first series of stimuli can be provided, in response to the first sleep disorder event, using a set of stimulus parameters. A habituation event can be detected and anti-habituation stimulation parameter can be adjusted to avoid patient habituation to the stimuli. The disclosures of these United States patents are incorporated herein by reference.

Patent application 20100076252 discusses, among other things, an apparatus and method for receiving respiration information of a patient and for providing stimulation to the patient using a hybrid circuit. The hybrid circuit includes a control input for receiving a control signal having a first and second state. A sense switch of the hybrid circuit can provide respiration information from a pyro/piezoelectric film sleep sensor and stimulator to a closed loop neuromodulator in response to the first state. A stimulation switch of the hybrid circuit can provide stimulation energy from the closed loop neuromodulator to the pyro/piezoelectric film sensor and stimulator in response to the second state. The hybrid circuit can couple to the pyro/piezoelectric film sensor and stimulator using a single wire pair. The disclosures of these United States patents are incorporated herein by reference.

### SUMMARY OF THE INVENTION

The Invention is directed to an apparatus and method for detecting, assessing and treating snoring and sleep apnea (Obstructive, Central, & Mixed) and hypopnea by terminating a snoring, sleep apnea event or hypopnea episode within seconds of detection.

The Invention develops through a method a referential set of parameters specific to the respiration patterns of the specific user (rather than defining and applying a generic trigger point parameter as is the case with other inventions). The multiplicity of signal parameters is more adaptable to the changes of respiration that occurs during the course of the night. Changes of respiration which might be interpreted by other inventions (such as those who use averaging or weighted moving averaging of respirations) as an indicator of an apnea event or hypopnea episode could be processed by this Invention and determined to be a reversion to a respiration pattern that is normal for the subject user. “Normal” respiration for the subject user is established by the processing of the referential set of parameters.

The Invention's method of using both the root-mean-square deviation of a parameter and the parameter's mean, as opposed to simply averaging or weighting the moving average of the parameter, to establish a reference point for determination of a parameter's out of bound condition, is a superior method for detecting, assessing and intervention of snoring, apnea events or hypopnea episodes.

In accordance with the Invention, there is provided a wearable apparatus for the detection, assessment and treatment of snoring, sleep apnea events and hypopnea episodes containing a microphone, pulse oximeter, and 3-axis accelerometer. The microphone and pulse oximeter generate signals that are representative of physiological aspects of respiration. The signals are transferred to an imbedded computer in the wearable apparatus and/or to a smart phone application. The embedded computer or smart phone application extracts the sound of breathing, blood oxygen level, heart rate and the user's sleeping position. The embedded computer and/or the smart phone application has the means for determining when respiration parameters falls out of defined boundaries for said respiration parameters. This method enables the real-time detection of the onset of snoring, a sleep apnea event or hypopnea episode. The embedded computer and/or a smart phone application supplies stimulation signals upon the detection of a snoring, sleep apnea event or hypopnea episode to initiate an inhalation or cessation of snoring. Said stimulation is provided in a manner so as to avoid the initiation of a cortical (EEG) arousal and vagal withdrawal of the parasympathetic tone to the heart. Said stimulus is delivered to the user by a cutaneous rumble effects actuator and/or audio effects broadcasting. Said actuator is embedded within a wearable component of the apparatus of said Invention.

It is a primary objective of the Invention to provide a system and method for detecting and terminating a snoring or sleep apnea event and hypopnea episode, within seconds of said detection, in a manner that will decrease or eliminate hypoxia, hypercapnia and the disturbance of pulmonary hemodynamics, and not awaken the user.

### BRIEF DESCRIPTION OF THE DRAWING(S)

The Invention will be described by reference to the following drawings, in which like numerals refer to like elements, and in which:

FIG. 1 A dimensional outline of the case of the Invention (Top view) of a preferred embodiment of the invention.

FIG. 2 A dimensional outline of the case of the Invention (Side view) of a preferred embodiment of the invention.

FIG. 3 A view of the bottom of the case of the Invention.

FIG. 4 A rendering of the Invention

FIG. 5 An illustration of the signals from respiration as received by the Inventions microphone.

FIG. 6 Is the Functional Block Diagram of the Invention.

FIG. 7 Is an illustration of a user wearing an embodiment of the Invention.

FIG. 8 Is an illustration of a user wearing an embodiment of the Invention.

### TECHNICAL PROBLEM

Positive Airway Pressure (PAP) systems are regarded as the most effective treatment for sleep apnea. Many patients, however, cannot tolerate the Positive Airway Pressure systems and associated apparatus, Common complaints include discomfort with the applied pressure, discomfort with the mask and equipment, nasal irritation, nasal stuffiness and congestion, airway dryness, mask air leaks and noise, entanglement, claustrophobia, noise of the PAP machine, headaches, abdominal bloating, sore and irritated eyes, and an overall discomfort with the machinery. The noise and general obtrusiveness of the PAP apparatus are often disruptive to another person sleeping with the user.

A significant percentage of the people for whom PAP is prescribed (estimated to be 30% to 50%) refuse to use it. A study determined that of the patients who use PAP treatment, it is estimated that 34% use it intermittently (4 nights per week) and/or remove it for part of the night (for this group median nightly usage is 3.1 hours).

Beyond the initial cost of the PAP (>US$800.00) and which has been categorized as having a lifetime of 5 years (per the guidelines for Durable Medical Equipment), there is the continuing cost of replacement masks. It is recommended that masks be replaced every six months (=>US$100.00/mask). Over a 5 year span the total costs of owning and using PAP could easily exceed US$1800.00.

A study determined that dental appliances were successful in treating OSA in an average of 52% of treated patients, with success defined as no more than 10 apneas or hypopneas per hour of sleep. Treatment adherence is variable with patients reporting using the appliance a median of 77% of nights at 1 year.

A dental appliance typically has a cost in excess of US$1500.00.

Surgery has inherent risks: cost is high, success rates vary and effectiveness fades over a period of time.

Pharmacological treatments for sleep apnea have not achieved an consistent levels of effectiveness, and often contain side effects.

Systems that clear the upper airway passages during sleep have been developed using the electrical stimulation of nerves or muscles. These systems may produce positive results but they also have associated risks due to surgery, may need replacement at later times (requiring additional surgery), and may have higher cost (it is expected that the price of the device and implantation surgery will cost approximately US$30,000.00) and have lower reliability than the more traditional treatments. In addition, the hybrid systems also have the accompanying physical restrictions and accompanying disadvantages associated with connections to the external equipment.

Oral pressure therapy (OPT) or continuous negative airway pressure (CNAP) is effective on approximately half of those with OSA. It requires a mouthpiece connected to an air tube and leaves the patient tethered to a bedside machine. It has an anticipated cost of US$1,400.00. This treatment ceases to be effective the moment the patient breathes through their mouth.

Expiratory Positive Airway Pressure (EPAP) is difficult to adjust to. It costs approximately US$60.00 per month and ceases to be effective the moment the patient breathes through their mouth.

An apparatus whose means for detecting the onset of a sleep related disorder that relies on blood oxygen content information cannot determine the onset of a sleep order in real time. Oxygen saturation level diminishment always lags the cessation of breathing because it takes time for the oxygen in the bloodstream to be used up by bodily processes. Hypoxia and hypercapnia will occur.

An apparatus whose sole means for detecting the onset of a sleep related disorder that relies on detecting the sounds of breathing can be confused by extraneous noises, coughing, wheezing and other internally generated biologic noises. In addition, in order for both the microphones and stimulus devices to work most effectively, they must be in close contact with the neck and this constriction may prove to be unacceptably uncomfortable to the user.

Many of these devices provide a single type of auditory stimulus a fixed tone of varying intensity) and/or mechanical stimulus (a vibrator) that the user may become habituated or accustomed to—reducing efficacy over time.

For example, U.S. Pat. No. 7,387,608 discloses such techniques. It is Claimed that: “The method of arousing the patient from sleep at the onset of a sleep apnea event will decrease or eliminate the occurrence of sleep apnea, arrhythmia, and partial epilepsy over time”

These methods of stimulus may prove to be initially effective in reducing the numbers of apnea events through a process of conditioning. However, with conditioning there co-exists habituation. These are two interacting psychological phenomena with a number of similarities. In conditioning, a test subject is exposed to some events, and as a consequence, it learns to associate a certain behavior with a specific situation. In habituation too, an event occurs repeatedly, but in this case, the reaction of the test subject wanes with repeated exposure.

The dynamics of habituation is very similar to the extinction of a response that has previously been learned during conditioning. In both cases, the response becomes less probable or weaker with each occurrence with the event. There is one large difference between the two situations, however. In extinction, a learned response is weakened, but in habituation the reaction that dies away is typically an innate orienting reaction. Conditioning may indeed lead to extinguishment of snoring and sleep apneas events or the opposite may occur; habituation might lead to the subject user ignoring the stimulus. If habituation occurs then snoring and sleep apnea events would continue until they spontaneously terminate.

### SOLUTION TO THE PROBLEMS

Therefore, there is a need in the art for an improved system and method for detecting, assessing and treating snoring, sleep apnea events and hypopnea episodes. In particular, there is a need in the art for an apparatus, system and method that does not create other types of sleep disturbing effects, does not require surgical implementation, does not involve the use of a complicated apparatus, does not include the use of pharmaceuticals, does not require the intervention of health professionals, and does not have the high costs associated with some of the types of assessments and treatments currently in use.

Therefore, there is a need for an apparatus, system and method for detecting, assessing and treating snoring and sleep apnea events and hypopnea episodes by terminating a snoring or sleep apnea event and hypopnea episode in real time that minimizes the disturbance to pulmonary hemodynamics.

And, there is a need for an apparatus, system and method for detecting, assessing and treating snoring, a sleep apnea event and hypopnea episode that is easy to use, comfortable, and less expensive than other methods of assessment and treatment.

### ADVANTAGEOUS EFFECTS OF THE INVENTION

An Advantageous Effect of Invention is the superior method of detection and assessment of apnea events and hypopnea episodes.

Using the standard deviation of a parameter in conjunction with the parameter's mean as opposed to using only a parameter's mean as a reference point for determination of a parameter's out of bound condition (excursion) leads to the diminishment of the occurrence of the Invention determining a false snoring, apnea event or hypopnea episode.

In the situation where the parameter's mean is the only reference, a single excursion beyond an established limit leads declaration of an apnea event or hypopnea episode. Conversely, with this method of the Invention, when an excursion is determined, a further determination is performed to establish if the excursion is smaller than every member of the set of parameters that were gathered during the self-calibration processes. For while an excursion might be smaller than the mean of the parameter that was calculated by the processes of the self-calibrations, it might be greater than any single parameter that formed the set of parameters that were determined to be “normal” for the subject user and which formed the reference set of parameters.

The Invention analyzes a multiplicity of parameters derived from redundant apparatus to detect respirations.

Another Advantageous Effect of the Invention is the ability to determine if a user has Positional OSA and to intercede such that it will cause the user to move to a non-supine sleeping position. It has been determined through research that approximately over half (56%) of all patients with Obstructive Sleep Apnea Syndrome (OSAS) have Positional OSA, where the number of supine apneas are >2× greater than non-supine apneas. This is especially the case with those who suffer from light or moderate OSA.

The 3-axis accelerometer determines the position of the user (supine, lateral, or dorsal). This positional information is evaluated by the processor embedded in the wearable apparatus or smart phone application in conjunction with the history of apneas (as determined by the processor embedded in the wearable apparatus or smart phone application from the audio signature of respirations collected by the microphone) to determine if user has Positional. OSA. If the Invention determines this to be true then the Invention will use plainly spoken phrases to cause the user to move from the supine position. This determination occurs only once and from that point on whenever the user used the Invention it will intervene.

Another Advantageous Effect of the Invention is the ability to differentiate between snoring, an apnea and a hypopnea. Each condition has a different treatment modality. An apnea requires an immediate stimulus to terminate it. A hypopnea's effect on blood oxygen levels and coronary/pulmonary systems takes a longer period of time to manifest. So long, that in many cases a subject will self-terminate the hypopnea before any dangerous physiological changes occur. The pulse oximeter data is evaluated by the processor embedded in the wearable apparatus and smart phone application which will determine if and when a stimulus is warranted to end the hypopnea.

Another Advantageous Effect of the Invention is its' ease of use. Many of the individuals who would use the Invention are both obese and old(er). The Invention is simple to don. The Invention uses plain language commands to guide the user how to properly position the invention.

Another Advantageous Effect of the Invention is it is not an encumbrance. The sleeping user is not physically constrained. This is important in light of the fact that many potential users have enlarged prostrates which, in many cases, necessitates frequent urination during the night.

Another Advantageous Effect of the invention is that it is less expensive that most other solutions. From the perspective of overall costs:
It does not require a costly sleep study or the programming of baseline parameters. Baseline parameters that have to be entered into an apparatus would require that there be an evaluation of the results from the user's polysomnography and using a method to establish baseline criteria. The Invention self determines the baseline parameters.
There are no replacement components. Other devices require periodic replacement of key components, at a considerable expense.
The Invention is far less expensive than the average price of the most popular assessment methods and forms of treatment for Obstructive Sleep Apnea—in-home tests or sleep lab studies, and CPAP machines.

Another Advantageous Effect of the invention is that it can be used in conjunction with the most popular form of treatment for Obstructive Sleep Apnea—CPAP—or as an alternate, independent form of treatment. There are a significant percentage of patients who use CPAP only intermittently. Using the Invention during those times that the user is not using CPAP would continue the benefit to the user that is obtained by maintaining normal blood oxygen and carbon dioxide levels.

Another Advantageous Effect of the Invention is it is self-adapting; it self-determines referential baselines for a subject user's normal respiration patterns. One of the definitions of Obstructive Sleep Apnea is interruptions in airflow of at least 10 seconds. The Invention may, depending on the normal respiration pattern of the subject user, establish a different baseline as to what an interruption of airflow in seconds would be.

By immediately applying a stimulus that has been determined to initiate an inhalation at the lowest level of stimulation, the effects on the physiology of the user of the snoring or apnea event, or hypopnea episode will be minimized.

Another Advantageous Effect of the Invention is that it applies the level of stimulus that was successful in terminating a previous apnea, unlike some Inventions that have devices that ramp up the stimulus (be it the frequency of a cutaneous rumble effects actuator and/or audio and/or amplitude) until respiration is restored. This takes time, in which case the deleterious effects of declining blood oxygen and increasing blood carbon dioxide accrue, and if it overshoots (there being a delay between the time a stimulus is applied and the reaction of the user to it) it could lead to a more heightened waking than is required to terminate the apnea event or hypopnea episode.

Another Advantageous Effect of the Invention is that it is self-adapting; it self-determines referential baselines for the type of stimulus that is required to terminate a snoring or apnea event or hypopnea episode. Research has shown that the amount of stimulus required to re-initiate inspirations changes in cycles during sleep. The Invention continuously evaluates the Stimulus required to terminate an apnea event or hypopnea episode.

Another Advantageous Effect of the Invention is that it can supply a very wide range of stimuli. It has a multiplicity of embedded (or stored on the smart phone application) audio files and haptic pattern files, each with a distinct irritation (level of arousal) index. The Invention will determine which files produce the stimulus required to initiate an inhalation at the lowest level stimulation. Since there are many file combinations that will produce the stimulus required to initiate an inhalation at the lowest level of stimulation, the Invention can avoid habituation while maintaining the benefit of conditioning,

### DESCRIPTION OF EMBODIMENTS

Accordingly, embodiments of the Invention are provided that meet least one or more of the following objects of the Invention.

In one embodiment the microphone collects the audio signature of respirations. These signals are evaluated by the processor embedded in the wearable apparatus or smart phone application which determines the quality of said respirations.

In another embodiment of the Invention, the pulse ox meter collects the blood oxygen saturation information of the user. This information is evaluated by the processor embedded in the wearable apparatus or smart phone application which determines the quality of said blood oxygen saturation level.

In another embodiment of the Invention, the Invention can determine if the User is going to have an apnea; this embodiment will be addressed in detail later in this document.

In another embodiment of the invention, the 3-axis accelerometer determines the sleeping positions (dorsal, supine, or side) of the user. This positional information is evaluated by the processor embedded in the wearable apparatus or smart phone application in conjunction with the history of apneas (as determined by the processor embedded in the wearable apparatus or smart phone application from the audio signature of respirations collected by the microphone) to determine if the user has positional apnea.

In another embodiment of the invention, a wireless auditory prompter (Bluetooth Earbud) is mounted in the user's ear and is activated by the stimulation signal to emit an acoustic stimulus which is heard by the user but is inaudible to others. This embodiment provides a sound to terminate snoring or initiate inhalation without requiring other intervention.

In another embodiment of the Invention, the processor embedded in the wearable apparatus communicates with the smart phone applications using a low power Bluetooth transceiver.

It another embodiment of the invention, a wired auditory prompter is mounted in the user's ear and is activated by the stimulation signal to emit an acoustic stimulus which is heard by the user but is inaudible to others. This embodiment provides a sound to terminate snoring or-initiate inhalation without requiring other intervention.

In another embodiment of the invention, a loud speaker is embedded within said. Invention and is activated by the stimulation signal to broadcast an acoustic stimulus which is heard by the user. This embodiment provides a sound to terminate snoring or initiate inhalation without requiring other intervention.

In another embodiment of the Invention, the processor embedded in the wearable apparatus or smart phone application detects the absence of a heartbeat and activates an audible alarm by said loud-speaker embedded within said. Invention.

In another embodiment of the Invention, the smart phone application has means to display information from a sleep cycle, inclusive of but not limited to
Date
Total hours of Sleep*
Total number of awakenings
Total number of snoring events
Total number of apneas
   Median length of apneas
   Average length of apneas
   Standard Deviation of apneas
Total number of hypopneas
   Median length of hypopneas
   Average length of hypopneas
   Standard Deviation of hypopneas
Total Time of SaO2 below 90%**
Total Time of Sa.O2 below 85%**
Total number of stimulus related arousals
   Median length of stimulus related arousals
   Average length of stimulus related arousals
   Standard Deviation of stimulus related arousals
Total number of non-stimulus related arousals
   Median length of non-stimulus related arousals
   Average length of non-stimulus related arousals
   Standard Deviation of non-stimulus related arousals
Sleeping position when snoring, apnea or hypopnea occurred

In another embodiment of the Invention, the processor embedded in the wearable apparatus or smart phone application has means to store the calculated amplitude, periodicity, and duration of respiration for each respiration of the collection of known good respirations from the first self-calibration in imbedded memory.

In another embodiment of the Invention, the processor embedded in the wearable apparatus or smart phone application has means to store the calculated values and parameters in imbedded memory.

In another embodiment of the Invention, the processor embedded in the wearable apparatus or smart phone application has means to store the time(s) in which a snoring, sleep apnea event and hypopnea episode occurs in imbedded memory.

In another embodiment of the Invention, the processor embedded in the wearable apparatus or smart phone application has means to store the time(s) in which a snoring, sleep apnea event and hypopnea episodes are terminated in imbedded memory.

In another embodiment of the Invention, the processor embedded in the wearable apparatus or smart phone application has means to export the calculated values and parameters from imbedded. memory to other devices.

In another embodiment of the Invention, the processor embedded in the wearable apparatus or smart phone application has means to export the time(s) in which a snoring, sleep apnea event and hypopnea episode occurs and from imbedded memory to other devices.

In another embodiment of the Invention, the processor embedded in. the wearable apparatus or smart phone application has means to export the time(s) in which a snoring, sleep apnea event and hypopnea episode are terminated from imbedded memory to other devices.

In another embodiment of the invention, the processor embedded in the wearable apparatus or smart phone application has means to import modifications of the computer programs from other devices.

In another embodiment of the Invention, there might be a plurality of microphones.

In another embodiment of the invention, the mechanical tactile sensory stimulator may be implemented using a Haptic Display.

In another embodiment of the Invention, the mechanical tactile sensory stimulator may be implemented using a Haptic Display comprising shape memory springs.

In another embodiment of the Invention, the mechanical tactile sensory' stimulator maybe implemented using a Haptic Display using multiple actuators.

In another embodiment of the invention, the mechanical tactile sensor stimulator maybe implemented using a Haptic Display comprising rotating drums.

In another embodiment of the Invention, the mechanical tactile sensory stimulator maybe implemented using a Haptic Display comprising electroactive polymers.

The foregoing description of the Invention have outlined the key features and technical advantages of the Invention so that those skilled in the art may better understand the detailed description of the Invention that follows. The foregoing descriptions are not meant to limit the claims of the Invention, but rather to describe the broad application of the various embodiments the Invention may take. Additional features and advantages of the Invention will be described hereinafter that form the subject of the claims of the Invention. Those skilled in the art should appreciate that they may readily use the conception and the specific embodiments disclosed as a basis for modifying or designing other structures for carrying out the same purposes of the invention. Those skilled in the art should also realize that such equivalent constructions or derivative works do not depart from the spirit and scope of the Invention in its broadest form.

Before undertaking the Detailed Description, definitions of certain words and phrases used throughout this patent document are set forth as follows: the terms “include” and “comprise” and derivatives thereof mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware, or software, or some combination of at least two of the same. Definitions for certain words and phrases are provided throughout this patent document. Those of ordinary skill in the art should understand that in many, if not most, instances, such definitions apply to prior, as well as future uses of such defined words and phrases.
“Computer”, “microcontroller”, “processor”, and “smart phone” are used interchangeably in this document and are collectively defined as the device that relies on the application of software programs that are resident within the computer as means or manner of procedure to accomplishing something. The means and reasons for said processing will be addressed in detail within document.
“Measurement” by the computer in this application is defined as an analog-to-digital conversion. The derivative of analog-to-digital conversion is a numeric value that is representative of the signals amplitude at the time that the measurement is made. Those skilled in the art will understand the method of using analog-to-digital conversion.
“Processing”, “process”, “monitoring”, and “method” are used interchangeably in this document and are collectively defined as the application of software programs that are resident within the computer as means or manner of procedure to accomplishing something. The means and reasons for said processing will be addressed in detail within this document.
“Communication” is defined by as the method and processes by which the various electronic components pass data. to each another within and without (outside) the invention.
“Sensor”, “sensor head”, “wearable device” and “wearable apparatus” are defined as the element(s) of the Invention whereby respiration, blood oxygen level, heart rate and sleeping position are detected.

Other objects and features of the Invention will become apparent from the following detailed description considered in conjunction with the accompanying drawings. It is to be understood, however, that the drawings are designed solely for purposes of illustration and not as a definition of the limits of the Invention, for which reference should be made to the appended claims.

For a general understanding of the Invention, reference is made to the drawings. In the drawings, like reference numerals have been used throughout to designate identical elements.

In accordance with this Invention, there is provided an apparatus and method for the diagnosis and treatment of snoring, sleep apnea events and hypopnea episodes. In one embodiment of the Invention, the respirations of the user are monitored during sleep by the apparatus, which acts as a monitoring system to detect and treat snoring, sleep apnea events and hypopnea episodes in the user. The monitoring system is comprised of an integrated 3-axis accelerometer, an integrated pulse oximeter, an integrated microphone, integrated or external computer, a software program, apparatus and methods for applying stimulus to the user such as an integrated loud speaker, wired and wireless audio, and an integrated rumble effects actuator. The Invention is wearable, and is either attached directly to the skin using a non-allergenic medical grade adhesive patch, attached to the user's sleep clothing, a sensor head attached directly to the skin using a non-allergenic medical grade adhesive patch, and said device or sensor head is positioned on or close to the upper thorax of the user.

At the onset of a snoring, sleep apnea event or hypopnea episode the respiratory induced movement (expansion and contraction) of the thorax and or abdomen are significantly reduced. In addition, the movement of air into the lungs is significantly reduced. These decreases are indicators of an onset of a snoring, sleep apnea event or hypopnea episode. During sleep, it is normal for an individual's respiration parameters for amplitude, inhalation time, exhalation time, duration of respiration and the period to vary. Discerning between those normal variations in the parameters (for amplitude, inhalation time, exhalation time, duration of respiration and the period during sleep) and abnormal variations in parameters (for amplitude, inhalation time, exhalation time, duration of respiration and the period), is performed using a software program that compares those parameters gathered by monitoring sensors (for amplitude, inhalation time, exhalation time, duration of respiration and the period during sleep) to those parameters (for amplitude, inhalation time, exhalation time, duration of respiration and the period) gathered before the user fell asleep. This method accurately identifies the onset of a snoring, sleep apnea event or hypopnea episode and eliminates false determinations.

The computer's software program uses rules based processing to determine when stimulation is to be delivered to the user in order to restore airway patency (by inducing inspiration).

When the user's respiration parameters are determined by the rules based processing as showing the onset of a snoring or sleep apnea event, or hypopnea episode stimulation is delivered to the user.

The Invention may use historical data, software programs, algorithms or subroutines to assist with the determination of the rules based processing which are appropriate to the subject user.

The embedded computer's software program uses rules based processing to determine the least amount of stimulation required to induce inspiration.

The stimulation is in the form of acoustic audio signals and/or by a cutaneous rumble effects actuator. Rules based processing determines the least amount of stimulation required to induce inspiration.

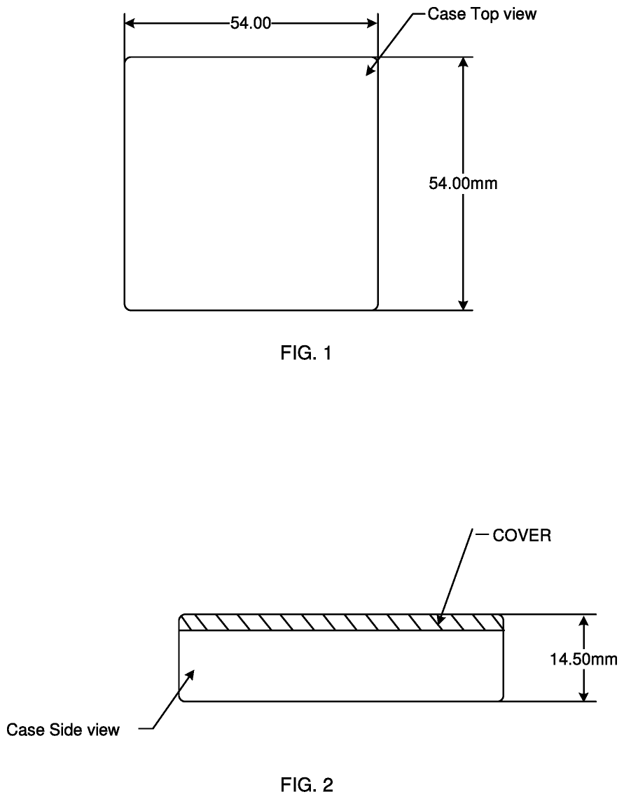

FIGS. 1 through 8, discussed below, and the various embodiments used to describe the principles of the Invention in this patent document are by way of illustration only and should not the construed in any way to limit the scope of the Invention. Those skilled in the art will understand that the principles of the Invention may be implemented in any suitably modified system for detecting, assessing and terminating a snoring or sleep apnea event or hypopnea episode.

FIG. 1 illustrates one embodiment of the Invention showing Sensor External view: Top

FIG. 2 illustrates one embodiment of the Invention showing Sensor External view: Side

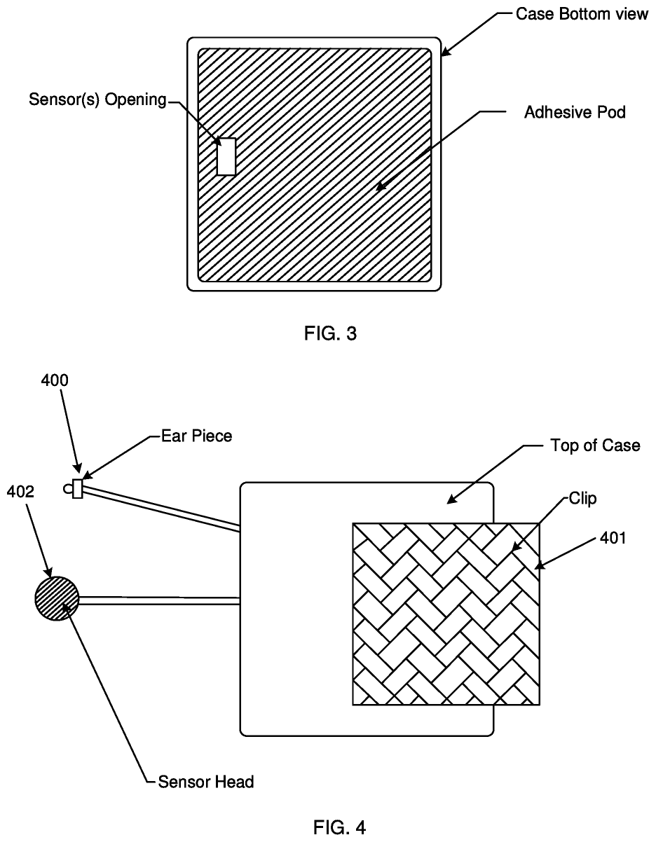

FIG. 3 illustrates one embodiment of the Invention showing Sensor External view: Bottom

FIG. 4 illustrates one embodiment of the Invention showing Sensor External view: Earbud 400, attachment Clip to attach the Sensor case to the user's bed clothing 401, Sensor head 402 which contains the LEDs/Photodetector of the pulse oximeter monitor section and the Microphone. The Sensor head is attached to the upper sternum with a non-allergenic medical grade adhesive pad.

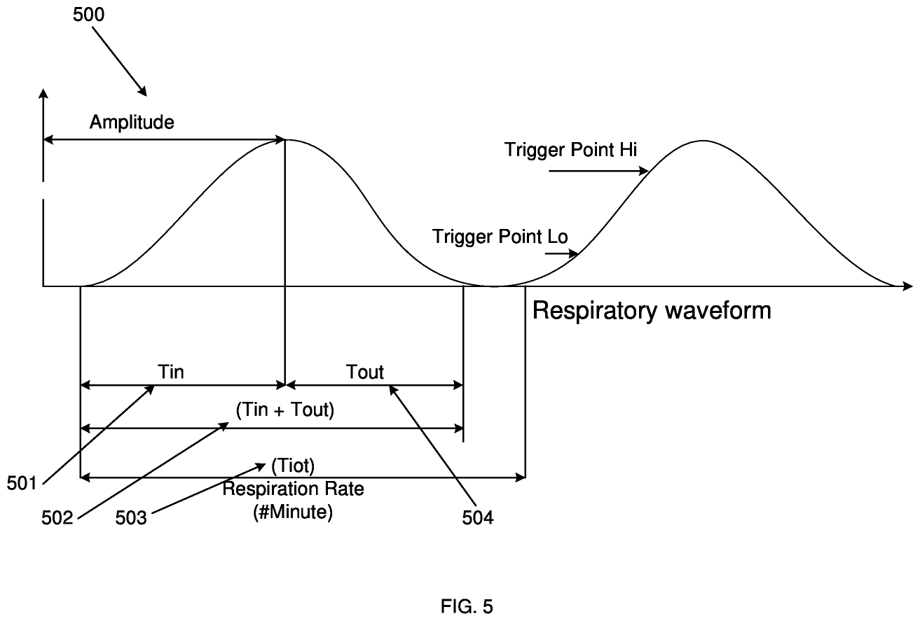

FIG. 5 is illustrative of the Signals of Breathing Sounds that is detected by Microphone (not shown in FIG. 5). The Microphone (not shown not shown in FIG. 5) detects a multiplicity of audio signals. The multiplicity of audio signals are comprised of the audio components of biologic processes (heart beats, audio component of the turbulence that occurs in the human respiratory system during respiration, bowels, snoring, wheezing, yawning, coughing, etc.) and external interference artifacts.

Referring again to FIG. 5: The Signals that are derived by the Microphone (not shown in FIG. 5) are measured by the Computer (not shown in FIG. 5). Each Signal is measured for five (5) discrete parameters. The measurement quantity is assigned a numeric value that represents a direct inferential reading of the specific Signal Parameter. The parameters that are measured are the:
Amplitude 500 of the Signal. The Amplitude 500 is representative of the expansion of the thorax or abdomen during an inspiration. It is a dimensionless number. The range is from 0 to 100.
Inhalation Time 501 (Tin) of the Signal; The amount time (in seconds) that it takes for a discrete inhalation.
Exhalation Time 504 (Tout) of the Signal: The amount time (in seconds) that it takes for a discrete exhalation
Duration of the Respiration (Tin+Tout) 502 of the Signal: The amount time (in seconds) that it takes for a discrete inspiration and exhalation to be completed.
Periodicity of the Signal 503 (Ttot): The amount of time (in seconds) from the start of the inhalation of a respiration to the start of the inhalation of the next respiration.

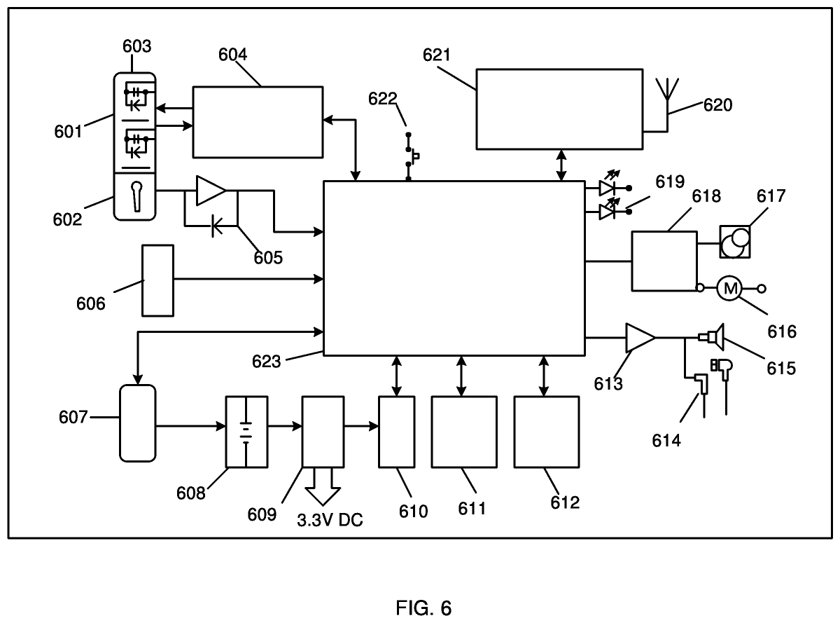

FIG. 6 Block diagram of the Electronic and Electrical elements of the Invention.

The operation of the Invention is illustrated in FIG. 6. It is made up of a number of electronic component modules:
601 is LEDs &Photodetector of the pulse oximeter sub system
602 is the microphone
603 is illustrative of one embodiment of the Invention wherein the 601 LEDs Photodetector of the pulse oximeter sub system and 602 the microphone are placed in separate enclosure; the Sensor head. The Sensor head 603 is attached to the user by a non-allergenic medical grade adhesive pad and transmits the signals that are picked up to the Sensor enclosure for processing.
604 is the analog front end of the pulse oximeter sub system
605 is the 100 Hz center frequency band pass filter
606 is the 3-axis accelerometer
607 is the USB port
608 is the Battery
609 is the Battery charger
610 is the Battery fuel tank
611 is the memory chip
612 is the Silicon Serial number
613 is the Audio amplifier
614 is the earbud
615 is the speaker
616 is the cutaneous rumble effects actuator
617 is the piezo-electric tactile stimulator/electroactive polymers
618 is the Haptic driver
619 are LEDs
620 is the Bluetooth transceiver antenna
621 is the Bluetooth transceiver
622 is the switch
623 is the microcontroller

FIG. 7 illustrates one embodiment of the Invention showing it positioned on the user's upper thorax.

FIG. 8 illustrates one embodiment of the Invention showing it positioned on the user's lower thorax.

### DETAILED DESCRIPTION OF THE INVENTION

FIG. 6 Block diagram. of the Electron c and Electrical elements of the Invention

It is a primary objective of the Invention to provide an apparatus and method for detecting, assessing and terminating a snoring or sleep apnea event and hypopnea episode, within seconds of said detection. To understand how said process is performed direct your attention to FIG. 6. FIG. 6 is a block diagram of the Block diagram of the Electronic and. Electrical elements of the Invention. LEDs & Photodetector 601 of the pulse oximeter sub system are used to detect the amount of the user's blood oxygen. The microphone 602 detects the sounds generated during respirations.

603 is illustrative of one embodiment of the Invention wherein the LEDs & Photodetector 601 of the pulse oximeter sub system and 602 the microphone are placed in separate enclosure—the Sensor head. The Sensor head 603 is attached to the user by a non-allergenic medical grade adhesive pad before sleep and transmits the signals that are picked up by the Sensor enclosure for processing. In another embodiment of the Invention wherein the LEDs & Photodetector 601 of the pulse oximeter sub system and the microphone 602 are placed within the single Sensor enclosure with the rest of the electronic devices as shown in FIG. 6 Block diagram of the Electronic and Electrical elements of the Invention

The analog front end (AFE) of the pulse oximeter sub system is shown in 604. The device consists of a low-noise receiver channel with an integrated analog-to-digital converter (ADC), an LED transmit section, and diagnostics for sensor and LED fault detection. The analog front end (AFE) 604 controls the LEDs & Photodetector 601 It is controlled by and communicates with the microcontroller 623.

Band Pass filter 605 accepts the audio signal generated by respirations and other bodily processes from microphone 602 (there is a multiplicity of audio signals which are comprised of the Audio components of biologic processes (heart beats, audio component of the turbulence that occurs in the human respiratory system during respiration, bowels, snoring, wheezing, yawning, coughing, etc.) and external interference artifacts)). The multiplicity of signals forms a spectrum of audio frequencies which are superfluous. Band Pass Filter 605 rejects (filters) all signals below 80 Hz and above 150 Hz. The remaining signal is that which is generated by respirations and is passed to the microcontroller 623 for further processing.

The 3-axis accelerometer is shown in 606. It generates signals that correspond to the X, Y, and Z axis of the user's body. Those signals are passed to the microcontroller 623 for further processing.

The USB port 607 serves a dual purpose. It serves as an axis point for external devices that communicate with the Invention. To accomplish that it is connected to the microcontroller 623. It also serves as an input point for power to recharge the Battery 608.

Battery 608 is a rechargeable lithium variant.

The Battery charger 609 monitors and charges the Battery 608. It communicates with the microcontroller 623.

The Battery fuel tank 610 performs measures the remaining power level of Battery 608. It communicates with the microcontroller 623.

Memory 611 contains the Processing program instructions as well as the results of computations performed by the microcontroller 623 and in one embodiment of the Invention, Audio files used for stimulus. It communicates with the microcontroller 623.

The Silicon Serial number 612 contains an electronic registration number that provides an absolutely unique identity for each copy of the Invention that is produced. It communicates with the microcontroller 623.

Audio Amplifier 613 provides the power to drive the Earbud 614 and Speaker 615 It communicates with the microcontroller 623.

Earbud 614 is a means to provide audio signals such as spoken directions and stimulus to the user.

Speaker 615 is a means to provide audio signals such as spoken directions and stimulus to the user.

Haptic Driver 618 provides the power to actuate the tactile motor 616 and the piezo-electric tactile stimulator/electroactive polymers 617. It communicates with the microcontroller 623.

The cutaneous rumble effects actuator 616 provides tactile stimulus to the user.

The piezo-electric tactile stimulator/electroactive polymers 617 provides tactile stimulus to the user.

LEDs 619 are Status indicators. The colors at the LEDs emit r indicative of operational conditions of the Tendon. They communicate with the microcontroller 623.

Antenna 620 is the means by which the Bluetooth Transceiver 621 sends and receives information to and from the smart phone application (not shown in FIG. 6), as well as sending provided audio signals such as spoken directions and stimulus to the user via a Bluetooth ear piece (not shown in FIG. 6) or the hardwired Earbud.

Bluetooth Transceiver 621 sends and receives information to and from the smart phone application (not shown in FIG. 6), as well as sending provided audio signals such as spoken directions and stimulus to the user via a Bluetooth ear piece (not shown in FIG. 6) or the hardwired Earbud It communicates with the microcontroller 623. Bluetooth is a wireless protocol utilizing short-range communications technology facilitating data transmission over short distances from fixed and/or mobile device. Bluetooth wireless communication is described, for example, in U.S. Pat. No. 7,225,064, issued to FUDALI, et al. May 29, 2007. The disclosures of these United States patents are incorporated herein by reference

Switch 622 controls the operations of the Invention. communicates with the microcontroller 623.

Microcontroller 623 receives information from the Sensor and transmits controlling signals to the electronic elements of the Invention as well as performing the processing of data/signals from the Smart Phone (not shown in FIG. 6) application and sensors.

Software Programs

The software programs are a series of modules. Each e is specific

to the task that it needs to perform.

Module 1: Initial Activation Aka Initialization Aka Self Check and Calibration

When the user first dons the Invention they will, in one embodiment of the Invention, insert the Earbud in their ear and, in one embodiment, activate the smart phone application. The user will then depress and release Switch 401.

In one embodiment this will establish the linkage between the Bluetooth Transceiver 621 and the smart phone application.

The purpose of that linkage is to use processing, storage (memory), and display capabilities of the smart phone.

In another embodiment of the Invention, there is no smart phone application so all processing and storage remains resident within the Sensor.

In all embodiments of the Invention, the device will give audio instruction for the user to enter an area that has the same background noise as the user's sleeping area. The Invention will calibrate itself to the user's hearing by applying a series of tones, starting with a tone of 0 dBM and ascending in 6 dBM steps until the user hears a tone. At that time the user will depress and release Switch 401. This will establish the lowest amplitude (loudness) signal that the user can discern. When an audio stimulus is applied, that determined value is the starting point for the amplitude (loudness) of the audio tiles that will be used. In this way the Invention has determined the range of hearing for that user.

The invention will ask the user to indicate if they are standing upright, If the user is doing so the user will depress and release Switch 401. This test is performed to determine if the 3-axis accelerometer is working correctly.

The Invention will instruct the User to silently count to 30. This test is performed to determine if the blood oximeter is working correctly as well as whether the microphone 602 and Band Pass filter 605 are receiving the audio signal generated by respirations and other bodily processes.

In addition, during this period of initialization the invention is computing and storing values of respirations: See FIG. 5.
Amplitude 500 of the Signal: The Amplitude 500 is representative of the expansion of the thorax or abdomen during an inspiration. The stored value is the peek amplitude for each respiration.
Inhalation Time 501 (Tin) of the Signal: The amount time that it takes for a discrete inhalation.
Exhalation Time 504 (Tout) of the Signal: The amount time that it takes for a discrete exhalation.
Duration of the Respiration (Tin+Tout) 502 of the Signal: The amount time that it takes for a discrete inspiration and exhalation to be completed.
Periodicity of the Signal 503 (Ttot): The amount of time from the start of the inhalation of a respiration to the start of the inhalation of the next respiration.

The Invention uses these values to constitute a set of respirations for the calculation of the Initial Referential Parameters. These initial Referential Parameters are calculated in the Invention to be 2 standard deviations (2 sigma) of the mean for each measured sets of the parameters. 95.45% of the values of a set for a parameter fall within 2 standard deviations (2 sigma) of the mean. The Initial Referential Parameters are stored in Memory 611.

The Invention now enters a time of hibernation which lasts until the user lies down.

Module 2: Full Activation

The Invention exits hibernation when the user lies down. This determination is made by monitoring and processing of the signals from the 3 Axis accelerometer 606.

The Invention now commences computing and storing values f respirations: See FIG. 5.
Amplitude 500 of the Signal. The Amplitude 500 is representative of the expansion of the thorax or abdomen during an inspiration. The stored value is the peek amplitude for each respiration.
Inhalation Time 501 (Tin) of the Signal: The amount of time that it takes for a discrete inhalation.
Exhalation Time 504 (Tout) of the Signal: The amount tinge that it takes for a discrete exhalation.
Duration of the Respiration (Tin+Tout) 502 of the Signal: The amount time that it takes for a discrete inspiration and exhalation to be completed.
Periodicity of the Signal 503 (Ttot): The amount of time from the start of the inhalation of a respiration to the start of the inhalation of the next respiration.

These values are placed into a 15 minute deep storage buffer in Memory 611. The buffer acts as a FIFO device; early values are replaced by later values on a first-in-first-out basis.

When the Invention determines that the user has fallen asleep it becomes active. In the active state it will intervene with stimulus as required and in a manner to terminate snoring, apneas or hypopneas that are causing a drop of oxygen blood level below approximately 94%. In addition if the user has been determined to have Positional OSA it will encourage the user to shift from a prone sleeping position with a verbal audio signal sent to Earbud 614 or Speaker 615. All these processes are addressed in detail later in this document.

The user is determined to be asleep when there is no peek respiration Amplitude 500<=30% of the Initial Referential Parameter for Amplitude 500 occurring within the time of the Initial Referential Parameter Periodicity of the Signal 503 (Ttot).

At that time a set of Final Referential Parameters are computed.

The Final Referential Parameters are derived by taking each of the values that were placed into the 15 minute deep storage buffer in Memory 611. Each set of those values (the five parametric values constitute a set) are compared to their Initial Referential Parameters. If any of the individual values are found to be either 25% greater than or less than it's Initial Referential Parameter, then the set is discarded.

This process continues with each set, working backwards from the newest to the oldest until 60 seconds of contiguous sets are found that meet the above criteria. Each parameter is then calculated to derive what is 2 standard deviations (2 sigma) of the mean for that parameter. 95.45% of the values of a set for a parameter fall within 2 standard deviations (2 sigma) of the mean. These derivations form the set of Final Referential Parameters.

This process is performed to determine what the normal parameters are for a sleeping subject user. What is “normal” varies from night to night. As a user sleeps their breathing patterns (and the derived parameters) change from when they are awake.

Module 3: Stimulation

It is a primary objective of the Invention to provide an apparatus and method for detecting, assessing and terminating a snoring or sleep apnea event and hypopnea episode, within seconds of said detection. To be specific it will determine if a user is snoring, if an apnea has occurred, if an apnea is going to occur, if the user has Positional OSA, if the user is supine, and if the user's blood oxygen has fallen below approximately 94%. This is done to decrease or eliminate snoring, hypoxia, hypercapnia and the disturbance of pulmonary hemodynamics in the user.

In each of these circumstances the Invention will supply an audio stimulus via the Earbud 614 or Speaker 615 or Haptic stimulus via the cutaneous rumble effects actuator 616 or the piezoelectric tactile stimulator/electroactive polymers 617.

A method of stimulation in one embodiment of the Invention is the playing of pre-recorded audio files. These audio files are stored in the Memory 611. Each audio file consists of a 3 second recording of sound with a specific content at a specific amplitude (loudness). Content is repeatedly re-recorded with a different amplitude (loudness). In this way the same content is in files with amplitudes that increase from 0 dB to 100 dB in 6 dB increments.

In keeping with a primary objective of the Invention, which is to interrupt snoring and restore patency (breathing) to the user as rapist as possible while avoiding awakening.

A user travels through 4 layers of sleep and REM. The deepest levels (3 & 4) and REM are considered to be the most restorative and are often referred to as slow-wave sleep. They are also the most difficult to arouse a person from. Therefore different stages of sleep might require different amplitudes (loudness) of audio stimuli.

Stimuli will often cause a change in sleep state (front deeper stage to a lighter stage); this is referred to as an arousal. Any application of any stimuli will always cause a change in blood pressure. The Invention applies stimuli in a manner so as to achieve these goals:
Interrupt snoring
Restore breathing as rapidly as possible
Minimize the length of time of an arousal (<3 s)
Cause the least amount of arousal (sub cortical arousal)
Minimize the change to the user's blood pressure and heart rate/pulse.

To accomplish the primary objective of the Invention the device will initially attempt an arousal using a randomly selected audio file with an amplitude (loudness) of that has the same amplitude as that which the user was able to discern during initialization. If snoring is terminated or patency is restored within 2 seconds (as defined by the detection of a respiration that is =>70% of Final Referential Parameter for Amplitude 500) then amplitude (loudness) of that audio file will become a referential starting point for the next time an audio stimulus needs to be applied. If that file is not successful in terminating snoring or restoring patency, then a random file that is louder will be applied. This process continues until snoring is terminated or patency is restored. Conversely if the Invention determines that files of the same amplitude (loudness) are repeatedly successful in restoring patency the next time an audio stimulus is applied it will be a random file with an amplitude that is less than that of the current referential starting point. If it is successful in restoring patency then the amplitude (loudness) of that audio file will become the new referential starting point. in this way the varying stages of sleep with their varying stimulus intensity requirements will be met.

A method of stimulation in another embodiment of the Invention is to provide tactile stimulus arousal.

The mechanical tactile sensory stimulator is the cutaneous rumble effects actuator 616. It differs from a simple vibrator in that it is capable of simulating a wide range of tactile effects, The Haptic effects are assembled by using software instructions to control the force amplitude, wave shape, and pulse duration to the stimulation effectors. These instructions are combined to form Force Portraits. The Force Portraits are stored in the Memory 611 in one embodiment of the invention. Different Force Portraits are felt as different tactile sensations by the user. These Force Portraits are assigned an Irritation Index value. Irritation Index values rank the relative irritation level as perceived by the user.

To accomplish the primary objective of the Invention the device will initially attempt an arousal using either the cutaneous rumble effects actuator 616 or the piezo-electric tactile stimulator/electroactive polymers 617. A randomly selected Force Portrait from the group of those Force Portraits with the lowest Irritation Index is initially applied. If snoring is terminated or patency is restored within 2 seconds (as defined by the detection of a respiration that is =>70% of Final Referential Parameter for Amplitude 500) then the irritation Index of that Force Portrait will become a referential starting point for the next time Force Portrait needs to be applied. If that Force Portrait is not successful in terminating snoring or restoring patency then a random Force Portrait that has the next larger Irritation Index will be applied. This process continues until snoring terminates or patency is restored. Conversely if the Invention determines that Force Portraits of the same Irritation Index are repeatedly successful in terminating snoring or restoring patency, the next time a Force Portrait is applied it will be a Force Portrait with an Irritation Index that is less than that of the current referential starting point. If it is successful in terminating snoring or restoring patency, then the Irritation Index of that Force Portrait will become the new referential starting point. In this way the varying stages of sleep with their varying stimulus intensity requirements can be met.

Those skilled in the art should understand that in order to prevent Habitation there are a large number of Force Portraits and. audio files that the invention can select from and apply.

Module 4: Detection, Assessment and Termination of Snoring

When the microphone 602 detects a respiration amplitude =>2× that of normal respiration amplitude for the user and that lasts longer that 3 seconds, a stimulus will be applied as described in Module 3 to arouse the user. If the user is determined to be sleeping in the supine position when microphone 602 detects a respiration amplitude =>2×. that of normal respiration amplitude for the user and that lasts longer that 3 seconds (such determination is made by the 3-axis accelerometer 606) and has been in the supine position for =>10 seconds, audio files, whose content consists of vocal admonishments of increasing urgency and amplitude (loudness) will be delivered to the Earbud 614 or the Speaker 615. The files will encourage the user to move from the supine position. The audio signal will increase in urgency and amplitude (loudness) until the user shifts sleeping position. The least urgent audio file with the lowest amplitude will be applied first, with a more urgent and greater amplitude audio file being played lithe user has not moved from the supine position with 5 seconds. This process continues, in a step like fashion, with an increasingly urgent and greater amplitude audio file being played until the desired result is achieved.

Module 5: Determination of an Apnea

If the Amplitude 500 for a Respiration is =<10% of the 2 sigma of the mean of the Final Referential Parameter for Amplitudes 500 (as computed with the time envelope of the 2 sigma of the mean of the Final Referential Parameter of Periodicity of the Signal 503 (Ttot)), then an apnea has occurred and the stimulus process starts.

Module 6: Determination of an Approaching Apnea

A Respiration signal whose Inhalation Time 501 (Tin) approaches 45% of the Periodicity of the Signal 503 (Ttot) for the Final Referential Parameter of Periodicity of the Signal 503 (Ttot), it is indicative of an approaching apnea. If this condition occurs then the stimulus process starts.

Module 7: Determination of a Hypopnea

The Invention will only apply a stimulus if the blood oxygen level (as measured by the integral pulse oximeter) falls below approximately 94%. The Invention will determine if a hypopnea is occurring if the Amplitude 500 for a Respiration is =<70% of the 2 sigma of the mean of the Final. Referential Parameter for Amplitudes 500 (as computed with the time envelope of the 2 sigma of the mean of the Final Referential Parameter of Periodicity of the Signal 503 (Ttot)) AND this condition continues for =>10 seconds.

Module 8: Determination of the Condition of Positional OSA

Apnea Hypoxia Index (AHI) is an index of sleep apnea severity that combines the numbers of apneas and hypopneas. Combining these gives an overall sleep apnea severity score that evaluates both number sleep disruptions and degree of oxygen saturation (blood oxygen level (as measured by the integral pulse taximeter). The AHI is calculated by dividing the total number of apnea and hypopnea events by the number of hours of sleep. (AHI values are typically categorized as 5-15/hr=mild; 15-30/hr=moderate; and >30/h=severe.).

Approximately over half (56%) of all patients with Obstructive Sleep Apnea Syndrome (OSAS) have Positional OSAS (POSAS), where the supine sleeping positions Apnea Hypoxia Index (AHI)>2× than the AH1 of those non-supine sleeping positions. This is especially the case with light or moderate OSAS. Various researchers have shown that the reduction of lying in the supine position is an effective treatment for positional sleep apnea. When the sleep position is adjusted, the symptoms and problems of positional sleep apnea reduce. The avoidance of lying supine is referred to as position therapy

The Invention will store the total number of occurrences of apneas and hypopneas during the second week of use by the user. The 3-axis accelerometer 606 will determine when the user is supine and not supine. Each incident of an apnea or hypopnea will be correlated with the sleeping position in which it occurred. At the end of the week the smart phone application or the embedded microprocessor will compute and compare the results of the data that accrued during that period. if the AHI for the supine sleeping position is greater than the AHI of the non-supine sleeping position then for the purposes of treatment by the Invention it will be determined that the user does have Positional OSA and hence forth the Invention will treat the user as having it.

Module 9: Treatment of Positional OSA

If the user is determined to be sleeping in the supine position (such determination is made by the 3-axis accelerometer 606) and has been in the supine position for =>10 seconds, audio files, whose content consists of vocal admonishments of increasing urgency and amplitude (loudness) will be delivered to the Earbud 614 or the Speaker 615. The files will encourage the user to move from the supine position. The audio signal will increase in urgency and amplitude (loudness) until the user shifts sleeping position. The least urgent audio file with the lowest amplitude will be applied first, with a more urgent and greater amplitude audio file being played if the user has not moved from the supine position with 5 seconds. This process continues, in a step like fashion, with an increasingly urgent and greater amplitude audio file being played until the desired result is achieved.

Module 10: The Awakening of the User Who does not Exit the Bed.

If the user is awoken by a stimulus the user will depress and release the Switch 622. The Invention will hibernate until it is determined by the Invention that the user has fallen back asleep. The user is determined to be asleep when there is no peek respiration Amplitude 500<=30% of the Initial Referential Parameter for Amplitude 500 occurring within the time of the Initial Referential Parameter Periodicity of the Signal 503 (Ttot). Upon determination that the user has gone to back to sleep the Invention will exit hibernation and fully activate.

Module 11: The Awakening of the User Who Sits Upright or Exits the Bed

If the 3-axis accelerometer 606 determines that the user is either sitting upright in bed or has exited the bed it will cause the Invention to hibernate until it is determined by the Invention that the user has fallen back asleep. The user is determined to be asleep when there is no peek respiration Amplitude 500<=30% of the Initial Referential Parameter for Amplitude 500 occurring within the time of the Initial Referential Parameter Periodicity of the Signal 503 (Ttot). Upon determination that the user has gone to back sleep of the Invention will exit hibernation and fully activate.

Module 12: The Awakening of the User (End of the Sleep Cycle)

When the microphone 602 ceases to acquire the audio signals associated with respirations the Invention will illuminate an LED 619 whose color will indicate the remaining charge of the Battery 608 for a period of 10 seconds. In addition, in one embodiment of the Invention a fuel gauge icon will be displayed on the smart phone application for 30 seconds indicating the battery state of discharge.

If the Battery 608 charge is low the user will plug a power source into the USB port 607 to recharge Battery 608

### CITATION LIST

### Patent Literature

US 2014/0051938 A1 Device to Detect and Treat Apneas and Hypopnea
U.S. Pat. No. 7,387,608 Apparatus and method for the treatment of sleep related disorders Jun. 1, 2008 Dunlop; David A, Gunderman, Jr.; Robert Dale
U.S. Pat. No. 7,371,220 System and method for real-time apnea/hypopnea detection using an implantable medical system May 1, 2008 Koh; Steve, Park; Euljoon, Benser; Michael
2005/0085865 Breathing disorder detection and therapy delivery device and method Apr. 1, 2005 Tehrani, Amir J
2006/0097879 SIDS and apnea monitoring system May 1, 2006 Lippincott; Kathy J
2005/0101833 Apparatus for the treatment apnea May 1, 2005 Hsu, William
U.S. Pat. No. 6,935,335 System and method for treating obstructive sleep apnea Oct. 1, 2005 Lehrman; Michael L., Halleck; Michael E
U.S. Pat. No. 6,666,830 System and method for detecting the onset of an obstructive sleep apnea event Dec. 1, 2003 Lehrman; Michael. L., Halleck; Michael E., Ferguson; Pete, Kumar; Harpal, Lay; Graham, Llewellyn; Mike, Place; John D.
U.S. Pat. No. 6,241,683 Phonospirometry for non-invasive monitoring of respiration Jun. 1, 2001 Macklem; Peter T, Que; Cheng-Li, Kelly; Suzanne M., Kolmaga; Krzystof, Durand; Louis-Gilles
U.S. Pat. No. 6,290,654 Obstructive sleep apnea detection apparatus and method using pattern recognition Sep. 1, 2001 Karakasoglu; Ahmet
U.S. Pat. No. 6,011,477 Respiration and movement monitoring system Jan. 4, 2000 Teodorescu; Horia-Nicolai, Mlynek; Daniel. J.
U.S. Pat. No. 5,853,005 Acoustic n monitoring system Dec. 1, 1998 Scanlon; Michael V
U.S. Pat. No. 5,769,084 Method and apparatus for diagnosing sleep breathing disorders Jun. 1, 1998 Katz; Richard A., Lawee; Michael S., Newman; A. Kief
U.S. Pat. No. 5,555,891 Vibrotactile stimulator system for detecting and interrupting apnea in infants Sep. 1, 1996 Eisenfeld; Leonard 1.
U.S. Pat. No. 5,540,733 Method and apparatus for detecting and treating obstructive sleep apnea Jul. 1, 1996 Testerman; Roy L., Erickson Donald J., Bierbaum; Ralph W.
U.S. Pat. No. 5,295,490 Self-contained apnea monitor Mar. 1, 1994 Dodakian; Wayne
U.S. Pat. No. 5,107,855 Apena monitor for detection of aperiodic sinusoidal movement Apr. 1, 1992 Harrington; Reginald, Crossley; Ralph
U.S. Pat. No. 5,050,614 Apparatus and method for inspiration detection Sep. 1, 1991 Logan; Charles H.
U.S. Pat. No. 4,781,201 Cardiovascular artifact filter Nov. 1, 1988 Wright; John C., Triebel; U.S. Pat. No. 4,694,839 Auxiliary stimulation apparatus for apnea distress Sep. 1, 1987 Timme; William F.
U.S. Pat. No. 4,686,999 Multi-channel ventilation monitor and method Aug. 1, 1987 Snyder; Leon. T., Scarfone; Frank A., Reuss; James L., Campen; George V., Yates; George H.
U.S. Pat. No. 4,365,636 Method of monitoring patient respiration and predicting apnea there from Dec. 1, 1982 Barker; Kent R.
U.S. Pat. No. 4,296,757 Respiratory monitor and excessive intrathoracic or abdominal pressure indicator Oct. 1, 1981 Taylor; Thomas Continuation Application Hypoxia Onset Detector And Interruptor
P20100076251 Pyro/Piezo Sensor at d Stimulator for Hybrid Circuit
P20100076251 Pyro/Piezo Sensor and Stimulator
P20100063350 Anti-habituating steep therapy for a closed loop neuromodulator
P20100063348 Stimulus sequencer for a closed loop neuromodulator
P20100057148 Stimulus tinier for a closed loop neuromodulator
P20100056855 Closed loop neuromodulator
P20100056852 Stimulus escalator for a closed loop neuromodulator
P20100048986 Dosage of optimization for a closed loop neuromodulator
P2009028765 Agitator to Stimulate the Central Nervous System

### Non Patent Literature

H. Schneider et al, “Effects of arousal and sleep state on systemanic and pulmonary hemodynamics in obstructive apnea”, J. Appl. Physiol, 88: 1084-1092, 2000.
D. M. Carlson et al, “Acoustically induced cortical arousal increases phasic pharyngeal muscle and diaphragmatic EMG in NREM sleep”, Journal of Applied Physiology, Vol 76, Issue 4 1553-1559.
W. T. McNicholas, “Arousal in the sleep apnea syndrome: a mixed blessing?”, Eur Respir J 1998; 12 1239-1241.
Robert C. Basner M D et al, “Respiratory and Arousal Responses to Acoustic Stimulation”, Chest. 1997; 112:1567-1571.
Gang Bao et al., “Acute and chronic blood pressure response to recurrent acoustic arousal in rats”, Am J Hypertens (1999) 12, 504-510.
R. C. Basner et al, “Effect of induced transient arousal on obstructive apnea duration”, J. App Physiol. 78(4):1469-1476, 1995.
Dina Brooks et al, “Obstructive Sleep Apnea as a Cause of Systemic Hypertension Evidence from a Canine Model”, J. Clin. Invest. Volume 99, Number 1, January 1997, 106-109.
Mary J. Morrell et al, “Sleep Fragmentation, Awake Blood Pressure, and Sleep-Disordered Breathing in a Population-based Study”, Am. J. Respir. Crit. Care Med., Volume 162, Number 6, December 2000, 2091-2096.
Robert C. Basner et al, “Respiratory and Arousal Responses to Acoustic Stimulation”, Chest 1997; 112; 1567-1571.
Richard S. T. Leung et al, “Sleep Apnea and Cardiovascular Disease”, Am. J. Respir. Crit. Care Med., Volume 164, Number 12, December 2001, 2147-2165.
Denise M. O'Driscoll et al, “Cardiovascular response to arousal from sleep under controlled conditions of central and peripheral chemoreceptor stimulation in humans”, J Appl Physiol 96:865-870, 2004.
Denise M. O'Driscoll et al, “Occlusion of the upper airway does not augment the cardiovascular response to arousal from sleep in humans”, J Appl Physiol 98:1349-1355, 2005.
U. Leuenberger et al, “Surges of muscle sympathetic nerve activity during obstructive apnea are linked to hypoxemia”, Am. J. Respir. Crit. Care Med., Volume 164, Number 12, December 2001 2147-2165.
Richard S. T. Leung et al, Sleep Apnea and Cardiovascular Disease”, Journal of Applied Physiology, Vol 79, Issue 2 581-588.
Richard B. Berry M D, “Sleep Apnea Impairs the Arousal Response to Airway Occlusion”, Chest. 1996; 109:1490-1496.
T. Kato et al, “Experimentally induced arousals during sleep: a cross-modality matching paradigm”, J. Sleep Res. (2004) 13, 229-23.
Christian Guilleminault et al, “The effect of CNS activation versus EEG arousal during sleep on heart rate response and daytime tests”, Clinical Neurophysiology 117 (2006) 731-739.
Emilia Sforza, M D et al, “Effects of Sleep Deprivation on Spontaneous Arousals in Humans”, SLEEP, Vol. 27, No. 6, 2004.
J. F. Masa et al, “Assessment of thoracoabdominal bands to detect respiratory effort-related arousal”, Fur Respir J 2003; 22: 661-667.
R. C. Basner et al, “Effect of induced transient arousal on obstructive apnea duration”, J Appl Physiol 78: 1469-1476, 1995.
Hiroshi Miki et al, “New Treatment for Obstructive Apnea Syndrome by Electrical Stimulation of Submental Region, Tohoku J. exp. Med., 1988, 154, 91-92.
C Guilleminault et al, “The effect of CNS activation versus EEG arousal during sleep on heart rate response and daytime tests”, Clinical Neurophysiology, Volume 117, Issue 4, Pages 731-739.
Immersion Corporation, “Next-generation TouchSense® Vibration for Video Game Console Systems”, 31 Aug. 2006.
Sheroz Khan, I. Adam et al, “Rule-Based Fuzzy Logic Controller with Adaptable Reference”, International Journal of Intelligent Technology Volume 3 Number 1.
E. Sforza et al, “Nocturnal evolution of respiratory effort in obstructive sleep apnea syndrome: influence on arousal threshold, Eur Respir j 1998; 121257-1263 DOI: 10.1183/09031936.98.12061257.
Homer Nazeran et al, “A Fuzzy Inference System for Detection of Obstructive Sleep Apnea”. Proceedings—23rd Annual Conference—IEEE/EMBS Oct. 25-28, 2001, Istanbul, TURKEY.

1-2. (canceled)

3. An apparatus for the detection, assessment, and treatment of sleep related disorders including snoring, sleep apnea, and hypopnea, the apparatus comprising:
one or more sensors configured for positioning proximal to a user and further configured to provide respiratory activity signals corresponding to said user's respiratory activity;
a user stimulator comprising at least one of a tactile stimulator, an audio effects broadcaster, a loudspeaker, a wireless earbud, or a hard-wired earbud; and
a processor configured to:
receive said respiratory activity signals;
assess said respiratory signals to detect and distinguish episodes of snoring, apnea, and hypopnea; and
in response to detecting an episode of snoring, apnea or hypopnea, provide a control signal to said user stimulator that causes the user stimulator to deliver a stimulus eliciting a sub-cortical arousal in the user;
wherein the stimulus delivered by the user stimulator is selected from a range of stimuli including nonstationary stimuli having a wide range of stimulating ability;
wherein a type, intensity, duration, and start time of the stimulus are chosen to treat the specific type of sleep disorder episode detected at the lowest effective level of stimulation;
wherein the type, intensity, duration, and start time of the stimulus are chosen to avoid or minimize recruitment of the ascending arousal system;
wherein the type, intensity, duration, and start time of the stimulus are chosen to avoid or minimize awakening the patient; and
wherein the type, intensity, duration, and start time of a stimulus selected to treat apnea are chosen to decrease sleep stages I and II and increase sleep stages III and IV.

4. The apparatus of claim 3, further comprising a pulse oximeter configured for positioning proximal to a user and further configured to provide pulse oximetry signals corresponding to said user's blood oxygen saturation and heart rate;
wherein the processor is further configured to:
receive said pulse oximetry signals;
assess said pulse oximetry signals together with said respiratory activity signals to detect and distinguish episodes of snoring, apnea, and hypopnea.

5. The apparatus of claim 3, further comprising a position and motion sensor configured for positioning proximal to a user and further configured to provide position and motion signals corresponding to said user's position and motion;
wherein the processor is further configured to:
receive said position and motion signals;
assess said position and motion signals together with said respiratory activity signals to detect and distinguish episodes of snoring, apnea, and hypopnea.

6. The apparatus of claim 3, wherein the processor employs rules-based processing.

7. The apparatus of claim 3, wherein the processor employs fuzzy logic.

8. The apparatus of claim 3, wherein the stimulus delivered by the user stimulator induces user inspiration.

9. The apparatus of claim 3, wherein the user stimulator is a tactile stimulator configured to engage a peripheral sensory area on said user.

10. The apparatus of claim 3, wherein the control signal is transmitted to the user stimulator via a wireless protocol.

11. A method for the detection, assessment, and treatment of sleep related disorders including snoring, sleep apnea, and hypopnea, the method comprising:
placing one or more sensors proximal to a user, the one or more sensors being configured to provide respiratory activity signals corresponding to said user's respiratory activity;
receiving said respiratory activity signals;
assessing said respiratory activity signals to detect and distinguish episodes of snoring, apnea, and hypopnea; and
in response to detecting an episode of snoring, apnea, or hypopnea, providing a control signal to a user stimulator that causes the user stimulator to deliver a stimulus eliciting a sub-cortical arousal in the user, the user stimulator comprising at least one of a tactile stimulator, an audio effects broadcaster, a loudspeaker, a wireless earbud, or a hard-wired earbud;
wherein the stimulus delivered by the user stimulator is selected from a range of stimuli including nonstationary stimuli having a wide range of stimulating ability;
wherein a type, intensity, duration, and start time of the stimulus are chosen to treat the specific type of sleep disorder episode detected at the lowest effective level of stimulation;
wherein the type, intensity, duration, and start time of the stimulus are chosen to avoid or minimize recruitment of the ascending arousal system;
wherein the type, intensity, duration, and start time of the stimulus are chosen to avoid or minimize awakening the patient; and
wherein the type, intensity, duration, and start time of a stimulus selected to treat apnea are chosen to decrease sleep stages I and II and increase sleep stages III and IV.

12. The method of claim 11, further comprising the steps of:
placing a pulse oximeter proximal to a user, the pulse oximeter being configured to provide pulse oximetry signals corresponding to said user's blood oxygen saturation and heart rate;
receiving said pulse oximetry signals;
assessing said pulse oximetry signals along with said respiratory activity signals to detect and distinguish episodes of snoring, apnea, and hypopnea.

13. The method of claim 11, further comprising the steps of:
placing a position and motion sensor proximal to a user, the position and motion sensor being configured to provide position and motion signals corresponding to said user's position and motion;
receiving said position and motion signals;
assessing said position and motion signals along with said respiratory activity signals to detect and distinguish episodes of snoring, apnea, and hypopnea.

14. The method of claim 11, wherein rules-based processing is employed to detect and distinguish episodes of snoring, apnea, and hypopnea.

15. The method of claim 11, wherein fuzzy logic is employed to detect and distinguish episodes of snoring, apnea, and hypopnea.

16. The method of claim 11, wherein the stimulus delivered by the user stimulator induces user inspiration.

17. The method of claim 11, wherein the user stimulator is a tactile stimulator configured to engage a peripheral sensory area on said user.

18. The method of claim 11, wherein the control signal is transmitted to the user stimulator via a wireless protocol.
